(19) United States
(12) Patent Application Publication
(10) Pub. No.: US 2007/0293943 A1
(43) Pub. Date: Dec. 20, 2007

(54) MEDICAL DEVICE SUITABLE FOR USE IN TREATMENT OF A VALVE

(75) Inventor: Martin Quinn, Blackrock (IE)

Correspondence Address: SUGHRUE MION, PLLC, 2100 PENNSYLVANIA AVENUE, N.W., SUITE 800, WASHINGTON, DC, 20037, US

(73) Assignee: MedNua Limited, Dublin (IE)

(21) Appl. No.: 11/763,590

(22) Filed: Jun. 15, 2007

Related U.S. Application Data
(60) Provisional application No. 60/813,694, filed on Jun. 15, 2006.

(51) Int. Cl. A61F 2/24

(52) U.S. Cl. 623/2.11; 623/2.36

(57) ABSTRACT

A medical device (1) suitable for use in treatment of a mitral valve comprises a treatment element (2) located at the region of co-aptation of the leaflets (3) of the mitral valve, a support element (4) which supports the treatment element (2) at the region of co-aptation of the valve leaflets (3), and an anchor element (8) to anchor the support element (4) to the ventricle wall at the apex (9) of the ventricle (5). The anchor element (8) is located at the distal end of the support element (4), and the proximal end (120) of the support element (4) is unconstrained relative to the wall of the ventricle (5) and the wall of the atrium (6). The treatment element (2) acts to resist blood flow in the retrograde direction through the valve opening.

This application claims benefit under 35 U.S.C. § 119(e) to U.S. Provisional Application No. 60/813,694 filed Jun. 15, 2006, the disclosure of which is incorporated herein by reference.

### INTRODUCTION

This invention relates to a medical device suitable for use in treatment of a valve, for example for use in treatment of the atrioventricular heart valves, and to a method of treating a valve.

The heart contains four valves, two semilunar, the aortic and pulmonary valves, and two atrioventricular (AV) valves, the mitral and tricuspid valves. The heart fills with blood from the lungs and body when the AV valves are open. When the heart pumps or contracts, the AV valves close and prevent the blood from regurgitating backwards. The semilunar valves open when the heart pumps allowing the blood to flow into the aorta and main pulmonary artery.

Dysfunction of the cardiac AV valves is common and can have profound clinical consequences. Failure of the AV valves to prevent regurgitation leads to an increase in the pressure of blood in the lungs or liver and reduces forward blood flow. Valvular dysfunction either results from a defect in the valve leaflet or supporting structure, or dilation of the fibrous ring supporting the valve. These factors lead to a failure of valve leaflets to meet one another, known as co-aptation, allowing the blood to travel in the wrong direction.

This invention is aimed at providing a medical device which addresses at least some of these problems.

### STATEMENTS OF INVENTION

According to the invention there is provided a medical device suitable for use in treatment of a valve, the device comprising:—
a treatment element configured to be located at the region of co-aptation of leaflets of a valve to resist fluid flow in a retrograde direction through an opening of the valve;
at least one support element to support the treatment element at the region of co-aptation of the valve leaflets; and
at least one anchor element to anchor the at least one support element to a wall of body tissue;
the at least one anchor element being located at the distal end of the at least one support element;
the proximal end of the at least one support element being unconstrained relative to the body tissue wall.

Because the proximal end of the support element is unconstrained, this arrangement facilitates a degree of lateral movement of the treatment element.

In one embodiment of the invention the support element is configured to extend through a valve opening. By extending at least part of the support element through the valve opening, this arrangement may facilitate location of the treatment element at the region of co-aptation of the valve leaflets extending through the valve opening. The support element may be dimensioned to extend, in use, from the anchor element through the interface between at least a pair of valve leaflets, to the treatment element. The anchor element may be extendable into a body tissue wall. The anchor element may be configured to extend only partially through a body tissue wall. The anchor element may be configured to be extended into a body tissue wall from an interior side of the body tissue wall. The anchor element may be configured to releasably anchor the support element to a wall of body tissue. The anchor element may comprise a threaded element. The threaded element may comprise a screw element. The anchor element may be configured to anchor the support element to a ventricle of a heart. The anchor element may be configured to anchor the support element to a septal wall of a ventricle of a heart. The anchor element may be configured to anchor the support element to the apex of a ventricle of a heart. The proximal end of the support element may be configured to be located externally of a heart.

In one embodiment of the invention the treatment element is movable between a collapsed configuration and an expanded configuration. The treatment element may be substantially tubular-shaped in the collapsed configuration. The treatment element may be substantially disc-shaped in the expanded configuration. The treatment element may be substantially curved in longitudinal cross-section in the expanded configuration. The convex portion of the curve may be configured to face towards leaflets of a valve. The convex portion of the curve may be configured to face distally. The treatment element may be substantially non-circular in lateral cross-section. The treatment element may be substantially elliptical in lateral cross-section. The major axis of the ellipse may be configured to be arranged substantially parallel to the major axis of a valve opening. The treatment element may be biased towards the expanded configuration. The treatment element may be at least partially of a shape-memory material.

In another embodiment of the invention a first end of the treatment element is movable relative to a second end of the treatment element to move the treatment element between the collapsed configuration and the expanded configuration. The first end may be movable and the second end may be configured to remain substantially stationary relative to leaflets of a valve. The first end may comprise the proximal end. The second end may comprise the distal end.

In one case the treatment element comprises a membrane.

In a further embodiment at least part of the treatment element is inflatable to move the treatment element from the collapsed configuration to the expanded configuration. The treatment element may comprise an inflatable region and a fluid passageway to connect the inflatable region in fluid communication with a fluid source. The treatment element may comprise one or more openings between the inflatable region and the fluid passageway.

In another case the treatment element is movable between a delivery configuration and a deployed configuration. In the delivery configuration the treatment element may have a larger radial dimension than in the deployed configuration. In the delivery configuration, the treatment element may be movable relative to the support element. In the deployed configuration, the treatment element may be fixed relative to the support element.

In one embodiment the treatment element comprises a collar member with a lumen extending therethrough. The collar member may be substantially circular in lateral cross-section.

In one case the treatment element is fixed to the support element. In another case the treatment element is movable relative to the support element for delivery of the treatment element over the support element to the region of co-aptation of leaflets of a valve.

In another embodiment the treatment element is mountable on the support element. The support element may comprise a mounting region upon which the treatment element is mountable, and a non-mounting region, the radial dimension of the mounting region being greater than the radial dimension of the non-mounting region. The treatment element may be engageable with the mounting region. The treatment element may be deliverable over the non-mounting region to the region of co-aptation of leaflets of a valve.

In one embodiment the support element is substantially flexible. The support element may comprise a wire element. The support element may comprise a pacing lead.

In another embodiment the device comprises a delivery member coupleable to the treatment element to facilitate delivery of the treatment element to the region of co-aptation of leaflets of a valve. The delivery member may comprise a delivery catheter for housing at least part of the treatment element. The device may comprise a release member to facilitate decoupling of the treatment element from the delivery member. The release member may be engageable with the treatment element to decouple the treatment element from the delivery member. The release member may be movable relative to the delivery member to decouple the treatment element from the delivery member.

According to another aspect of the invention there is provided a method of treating a valve, the method comprising the steps of:

locating a treatment element at the region of co-aptation of leaflets of the valve to resist fluid flow in a retrograde direction through an opening of the valve,

using at least one support element to support the treatment element at the region of co-aptation of the valve leaflets,

anchoring the distal end of the at least one support element to a wall of body tissue with the proximal end of the at least one support element being unconstrained relative to the body tissue wall.

In one embodiment of the invention the support element extends through the valve opening. The distal end of the support element may be extended into the body tissue wall. The distal end of the support element may be extended only partially through the body tissue wall. The distal end of the support element may be extended into the body tissue wall from an interior side of the body tissue wall. The distal end of the support element may be releasably anchored to the body tissue wall. The distal end of the support element may be anchored to a ventricle of a heart. The distal end of the support element may be anchored to a septal wall of a ventricle of a heart. The distal end of the support element may be anchored to the apex of a ventricle of a heart. The proximal end of the support element may be located externally of a heart.

In one case the method comprises the step of moving the treatment element between a collapsed configuration and an expanded configuration. A first end of the treatment element may be moved relative to a second end of the treatment element to move the treatment element between the collapsed configuration and the expanded configuration. The first end may be moved and the second end may remain substantially stationary relative to the valve leaflets. At least part of the treatment element may be inflated to move the treatment element from the collapsed configuration to the expanded configuration.

In another embodiment the method comprises the step of moving the treatment element between a delivery configuration and a deployed configuration. The method may comprise the step of moving the treatment element in the delivery configuration relative to the support element. In the deployed configuration, the treatment element may be fixed relative to the support element.

In another case the treatment element is fixed to the support element, and the method comprises the step of advancing the support element to deliver the treatment element to the region of co-aptation of the valve leaflets.

In one embodiment the method comprises the step of moving the treatment element over the support element to deliver the treatment element to the region of co-aptation of the valve leaflets.

In one case the method comprises the step of mounting the treatment element to the support element. The treatment element may be engaged with the support element to mount the treatment element to the support element.

In another embodiment the method comprises the step of coupling the treatment element to a delivery member before delivery of the treatment element to the region of co-aptation of the valve leaflets. At least part of the treatment element may be housed within the delivery member. The method may comprise the step of decoupling the treatment element from the delivery member at the region of co-aptation of the valve leaflets.

The treatment element may act as a support to at least partially support at least one valve leaflet at the region of co-aptation of the valve leaflets. The treatment element may act as an occluder to at least partially occlude a valve opening.

By supporting the valve leaflets at the region of co-aptation and/or occluding the valve opening, the medical device of the invention may be suitable for use in treatment of a number of defects in an atrioventicular valve, such as valve prolapse, or annular dilation of a valve, or restriction of a valve.

In one case the device is configured for use in treatment of a unidirectional valve. The treatment element may be configured to facilitate fluid flow in a forward direction through a valve opening.

In one embodiment the treatment element, when deployed, is shaped and dimensioned to permit unidirectional flow of fluid therepast.

The treatment element may be configured to be urged towards a valve opening by fluid flow. The treatment element may be shaped to be urged towards a valve opening by fluid flow. By arranging the fluid flow to urge the treatment element towards the valve opening, this arrangement may assist in preventing the treatment element from moving into the ventricle by an excessive amount or fully into the ventricle. The treatment element may be configured wherein fluid flow urges the treatment element in a direction from the ventricle towards the atrium.

A crescent shape for the treatment element may be particularly suitable for use with a mitral valve which has a normally crescent shaped opening.

The treatment element may be formed in a range of dimensions to suit the particular anatomy of a patient.

In one case the treatment element is engageable with at least one leaflet of a valve.

In the expanded configuration the treatment element may be engageable with a valve leaflet. In the expanded configuration the treatment element may be sealingly engageable with a valve leaflet. In the collapsed configuration the treatment element may be deliverable through a vasculature to a treatment site.

In one case the treatment element is engageable with a valve leaflet which is movable between a closed configuration and an open configuration. In the closed configuration the treatment element may be engageable with a valve leaflet. In the closed configuration the treatment element may be sealingly engageable with a valve leaflet. The treatment element may comprise a plug element. In the closed configuration the treatment element may be configured to prevent fluid flow through a valve opening. In the open configuration the treatment element may be spaced-apart from the region of co-aptation of the valve leaflets. In the open configuration the treatment element may be configured to resist fluid flow in the retrograde direction through a valve opening. In the open configuration the treatment element may be configured to facilitate fluid flow in the forward direction through a valve opening.

In one case the treatment element is engageable with a valve leaflet at an engagement region spaced substantially from an annulus of the valve. The treatment element may be engageable with a valve leaflet at the region of co-aptation of the valve leaflets. The treatment element may be engageable with a valve leaflet at an engagement region in proximity to or within the valve opening.

In one case the treatment element is configured to be located adjacent an interface between at least a pair of valve leaflets. The treatment element may be configured to at least partially prevent leakage from the interface.

The support element may be configured to support the treatment element in a location adjacent to a valve opening. The support element may be configured to support the treatment element in a location externally of a valve opening. The support element may be configured to support the treatment element extending at least partially through a valve opening.

It will be appreciated that movement of the heart, for example during the cardiac beating cycle, may result in the treatment element moving relative to the valve leaflets. By extending the treatment element at least partially through the valve opening, this arrangement may result in a degree of redundancy to ensure that at least part of the treatment element is located at the region of co-aptation of the valve leaflets at all times.

In another arrangement, the treatment element may be located adjacent to a valve opening, externally of the valve opening and not extending through the valve opening.

In one case the treatment element is carried on the support element.

In one case the anchor element comprises a hook element. The anchor element may comprise a suture loop.

The position at which the treatment element may be located along the support element may be varied.

The support element may have sufficient torsional rigidity to enable the support element to be used to screw the anchor element to a wall of a heart.

In one embodiment the device comprises a delivery system to facilitate delivery of the treatment element to the region of co-aptation of the valve leaflets. The delivery system may comprise a percutaneous delivery system to facilitate percutaneous delivery of the treatment element to the region of co-aptation of the valve leaflets.

In one case the treatment element at least partially comprises a shape-memory material. The shape-memory material may comprise nitinol.

In another case the treatment element is collapsible to facilitate delivery of the treatment element via a sheath or the like. The treatment element may be dimensioned when collapsed, to facilitate percutaneous delivery of the treatment element.

The treatment element of the medical device may be deployed using minimally invasive techniques. In particular it may be possible to deliver the treatment element to the region of co-aptation of the valve leaflets, and securely support the treatment element at the region of co-aptation using percutaneous techniques.

In one embodiment the treatment element is at least partially comprised of a resiliently deformable material. The configuration of the treatment element may be adjustable in-situ at the region of co-aptation of the valve leaflets. The size of the treatment element may be adjustable in-situ. The radial dimension of the treatment element may be adjustable in-situ.

The device may be configured for use in treatment of a heart valve. The device may be configured for use in treatment of an atrioventricular valve. The device may be configured for use in treatment of a mitral valve or a tricuspid valve. The treatment element may be configured to be located in an atrium of a heart. The treatment element may be configured to be located extending from an atrium of a heart at least partially through a mitral valve or a tricuspid valve.

As used in this patent specification, the term “interface” will be understood to mean an area at which two elements or surfaces meet or approach one another without necessarily touching.

As used in this patent specification, the term “plug” will be understood to mean a component or collection of components which are adapted to at least partially fill or occlude a gap between two or more surfaces or the like, whether using the whole plug or a portion thereof.

As used in this patent specification, the term “repair” will be understood to mean the procedure of resisting retrograde fluid flow through a valve, for example by at least partially supporting at least one of the valve leaflets at the region of co-aptation of the valve leaflets and/or by at least partially occluding the valve opening.

### BRIEF DESCRIPTION OF THE DRAWINGS

The invention will be more clearly understood from the following description of some embodiments thereof, given by way of example only, with reference to the accompanying drawings, in which:—

FIG. 1 is a partially cross-sectional, side view of a medical device according to the invention, in use;

FIG. 2 is an end view of the device of FIG. 1;

FIGS. 3 and 4 are partially cross-sectional, side views of the device of FIG. 1, in use;

FIG. 5 is an end view of the device of FIG. 1, in use;

FIGS. 6 to 10 are cross-sectional, side views of another medical device according to the invention, in use;

FIGS. 11 to 13 are cross-sectional, side views of another medical device according to the invention, in use;

FIGS. 14 to 18 are cross-sectional, side views of a further medical device according to the invention, in use;

FIG. 19 is an isometric view of the device of FIG. 18;

FIG. 20 is a cross-sectional, side view of another medical device according to the invention;

FIGS. 21 to 29 are cross-sectional, side views of the device of FIG. 20, in use;

FIGS. 30 to 36 are cross-sectional, side views of another medical device according to the invention, in use; and

FIGS. 37 to 40 are side views of support elements of other medical devices according to the invention.

### DETAILED DESCRIPTION

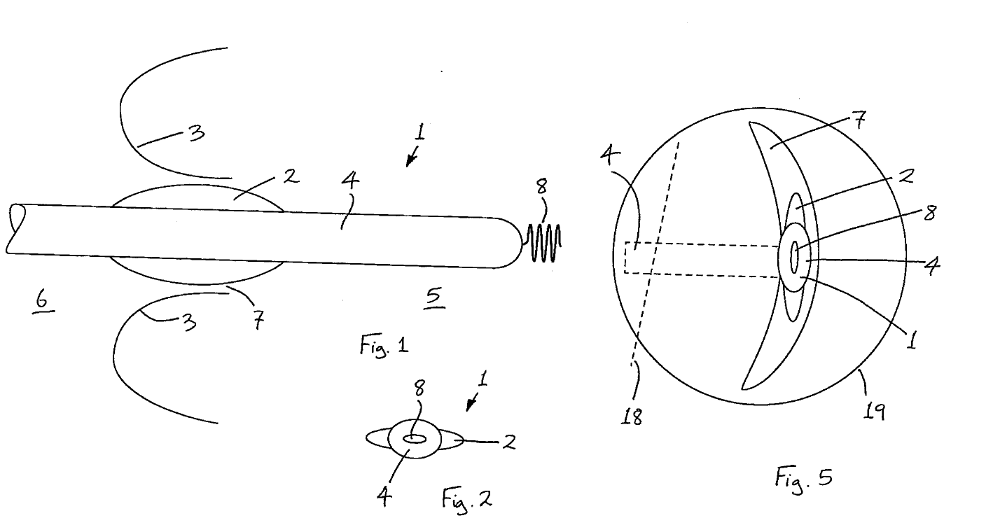

Referring to the drawings, and initially to FIGS. 1 to 5 thereof, there is illustrated a medical device 1 according to the invention. The device 1 is suitable for use in treatment of a valve, for example one of the atrioventricular heart valves.

The device 1 comprises a treatment element 2 which is configured to be located at the region of co-aptation of the leaflets 3 of the atrioventricular heart valve, and a support element 4 which supports the treatment element 2 at the region of co-aptation of the valve leaflets 3 (FIG. 1).

The treatment element 2 acts to resist blood flow in the retrograde direction from the ventricle 5 into the atrium 6 through the valve opening 7.

In this case the support element 4 is provided in the form of a flexible wire, for example a pacing lead. The support element 4 extends through the valve opening 7, in use.

The treatment element 2 is fixedly attached to the support element 4. The support element 4 is advanced, in use, to deliver the treatment element 2 to the region of co-aptation of the valve leaflets 3.

As illustrated in FIG. 2, the treatment element 2 has an elliptical shape in lateral cross-section, and the support element 4 has an elliptical shape in lateral cross-section. The major axis of the treatment element ellipse is greater than the major axis of the support element ellipse. The minor axis of the treatment element ellipse is less than the minor axis of the support element ellipse. The elliptical shapes of the treatment element 2 and of the support element 4 are particularly suitable for treating the mitral valve which has a crescent-shaped opening 7, as illustrated in FIG. 5. The major axis of the treatment element ellipse is arranged parallel to the major axis of the crescent-shaped valve opening 7, in use (FIG. 5).

The device 1 also comprises an anchor element 8 located at the distal end of the support element 4. The anchor element 8 comprises a threaded screw. The anchor element 8 may be releasably attached to the ventricle septal wall at the apex 9 of the ventricle 5, for example by screwing the anchor element 8 into the ventricle wall. In this manner the support element 4 will be anchored to the ventricle wall and the treatment element 2 will be maintained in the desired position relative to the valve leaflets 3. The anchor element 8 extends only partially through the ventricle wall from the interior side of the ventricle wall.

The proximal end 120 of the support element is unconstrained relative to the wall of the ventricle 5 or the wall of the atrium 6. The proximal end 120 of the support element 4 is located externally of the heart, in use.

In use, the support element 4 is advanced through the atrium 6, through the valve opening 7, and into the ventricle 5 until the treatment element 2 is located at the region of co-aptation of the valve leaflets 3. The support element 4 is then rotated to screw the anchor element 8 into the ventricle wall at the apex 9 of the ventricle 5. The treatment element 2 is thus supported in the desired location to treat the valve.

If it is desired to remove the device 1, the support element 4 is rotated to unscrew the anchor element 8 from the ventricle wall. The support element 4 is then withdrawn from the ventricle 5 through the valve opening 7, and withdrawn from the atrium 6.

FIG. 1 illustrates the pacing lead 4 fixed in the left ventricle 5 with the expansion 2 at the level of the mitral valve. FIG. 2 illustrates an end on view.

FIG. 3 illustrates the device 1 positioned across the mitral valve orifice 7, the right atrium 10, the tricuspid valve 11, the papillary muscle 12, the right ventricle 13, the left ventricle 5, the chordae tendiniae 14, the mitral valve, and the left atrium 6.

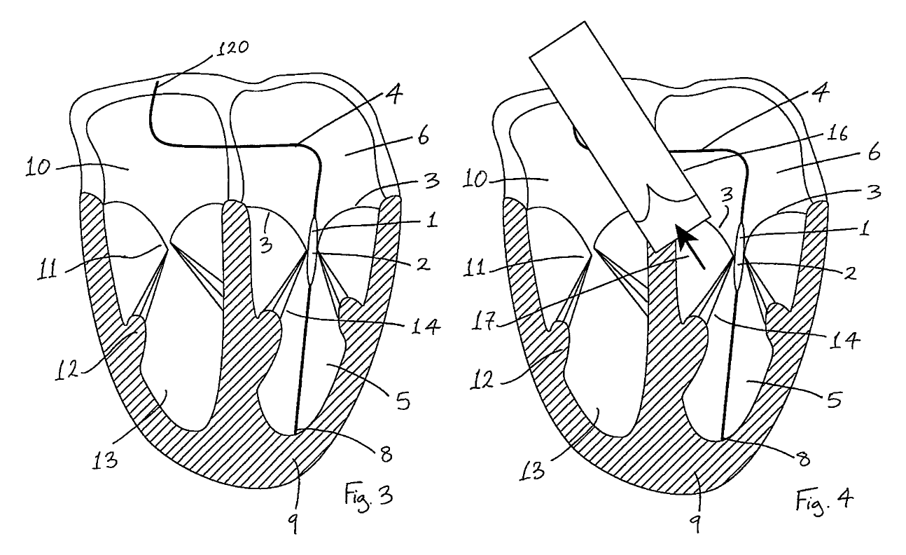

FIG. 4 illustrates the relationship to the aortic valve 16, the direction 17 of blood flow during systole, the device 1, the mitral valve, and the chordae tendiniae 14.

FIG. 5 illustrates the treatment element 2 positioned in the mitral valve orifice 7, looking from the apex 9 into the heart, the proximal end of the lead 4 in the left atrium 6 crossing the atrial septum, the atrial septum 18, and the mitral valve ring 19.

In FIGS. 6 to 10 there is illustrated another medical device 20 according to the invention, which is similar to the device 1 of FIGS. 1 to 5, and similar elements in FIGS. 6 to 10 are assigned the same reference numerals.

In this case the treatment element is provided in the form of a collar member 21 which is substantially circular in lateral cross-section (FIG. 10). The collar member 21 has a lumen 24 extending therethrough. The collar member 21 is formed separately from the support element 4.

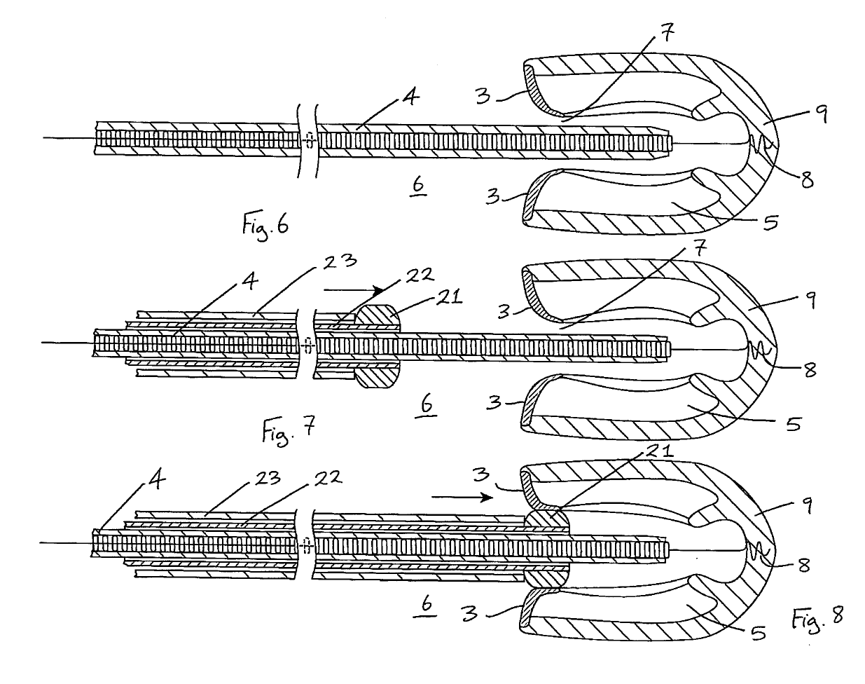

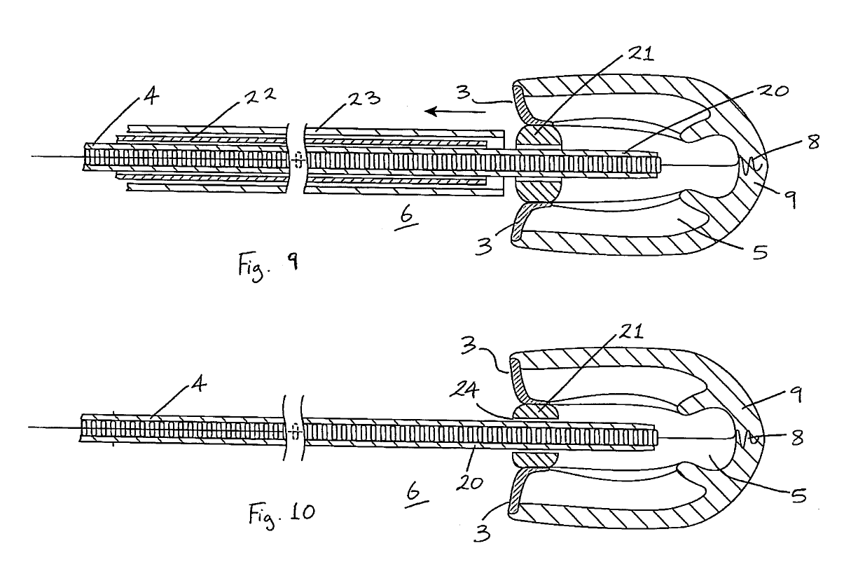

The collar member 21 is movable between a delivery configuration (FIGS. 7 and 8) and a deployed configuration (FIGS. 9 and 10). In the delivery configuration, the collar member 21 has a larger radial dimension than in the deployed configuration. In the delivery configuration, the collar member 21 is movable relative to the support element 4 to facilitate delivery of the collar member 21 over the support element 4 to the region of co-aptation of the valve leaflets 3 (FIGS. 7 and 8). In the deployed configuration, the collar member 21 is fixed relative to the support element 4, for example by being clamped to the support element 4.

In this case a delivery catheter 22 is provided coupled to the collar member 21 for delivery of the collar member 21 to the region of co-aptation of the valve leaflets 3. The delivery catheter 22 maintains the collar member 21 in the delivery configuration until the collar member 21 reaches the region of co-aptation of the valve leaflets 3. A release member 23 is movable distally relative to the delivery catheter 22 to engage the collar member 21 to decouple the collar member 21 from the delivery catheter 22, and thus release the collar member 21 to move from the delivery configuration to the deployed configuration.

In use, the support element 4 is advanced through the atrium 6, through the valve opening 7, and into the ventricle 5 until the anchor element 8 reaches the apex 9 of the ventricle 5. The support element 4 is then rotated to screw the anchor element 8 into the ventricle wall at the apex 9 of the ventricle 5 (FIG. 6).

The delivery catheter 22 with the collar member 21 in the delivery configuration is advanced over the support element 4 (FIG. 7) until the collar member 21 reaches the region of co-aptation of the valve leaflets 3 (FIG. 8). The release member 23 is then moved distally relative to the delivery catheter 22 to release the collar member 21 to move from the delivery configuration to the deployed configuration clamped to the support element 4 (FIG. 9). The delivery catheter 22 and the release member 23 are withdrawn from the atrium 6 (FIG. 10).

FIG. 6 illustrates the pacing lead 4 screwed into the ventricle wall.

FIG. 7 illustrates the delivery catheter 22 with the shaped co-aptation collar 21 mounted at the distal end. The implant 21 is expanded following delivery. The delivery catheter 22 may be rapid exchange or over the wire (OTW).

FIG. 8 illustrates the implant 21 delivered to the valve region and positioned. The collar 21 is frictionally mounted on the inner tube 22. The outer tube 23 is used to deploy the collar 21.

FIG. 9 illustrates the outer tube 23 held firm while the inner tube 22 is withdrawn. The collar 21 contracts and becomes attached to the shaft of the pacing lead 4.

FIG. 10 illustrates the delivery catheter 22 removed and the collar 21 left in situ.

FIGS. 11 to 13 illustrate another medical device 30 according to the invention, which is similar to the device 20 of FIGS. 6 to 10, and similar elements in FIGS. 11 to 13 are assigned the same reference numerals.

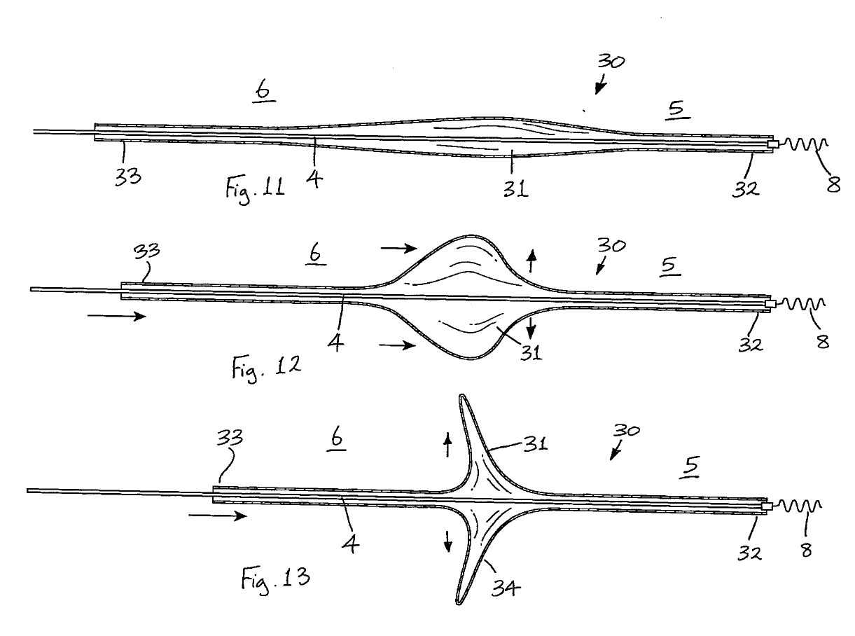

In this case the treatment element 31 is provided in the form of a membrane. The treatment element 31 is movable between a collapsed delivery configuration (FIG. 11) and an expanded deployed configuration (FIG. 13). The treatment element 31 is movable from the collapsed configuration to the expanded configuration by maintaining the distal end 32 of the treatment element 31 in a substantially fixed position relative to the valve leaflets 3 and moving the proximal end 33 of the treatment element 31 distally.

The treatment element 31 is substantially tubular-shaped in the collapsed configuration (FIG. 11).

In the expanded configuration, an intermediate portion 34 of the treatment element 31 is substantially disc-shaped (FIG. 13). The intermediate portion 34 is curved in longitudinal cross-section with the convex portion of the curve facing distally towards the valve leaflets 3.

In use, the treatment element 31 is advanced over the support element 4 until the intermediate portion 34 of the treatment element 31 reaches the region of co-aptation of the valve leaflets 3. The distal end 32 of the treatment element 31 is maintained in a substantially fixed position relative to the valve leaflets 3, and the proximal end 33 of the treatment element 31 is moved distally to move the treatment element 31 from the collapsed configuration to the expanded configuration.

FIGS. 11 to 13 illustrate the pacing lead 4 with the regurgitation collar 34.

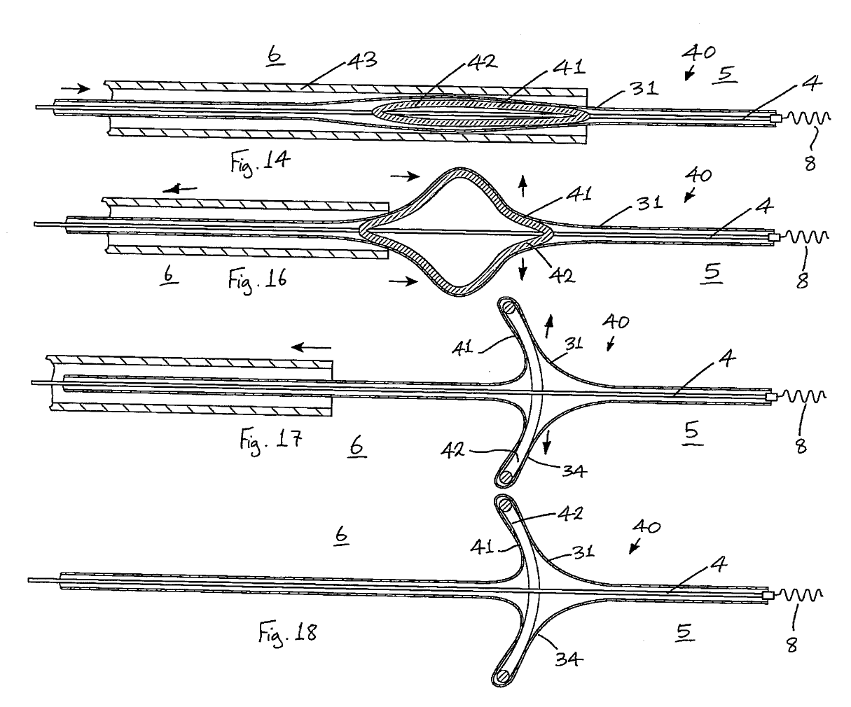

Referring to FIGS. 14 to 19 there is illustrated another medical device 40 according to the invention, which is similar to the device 30 of FIGS. 11 to 13, and similar elements in FIGS. 14 to 19 are assigned the same reference numerals.

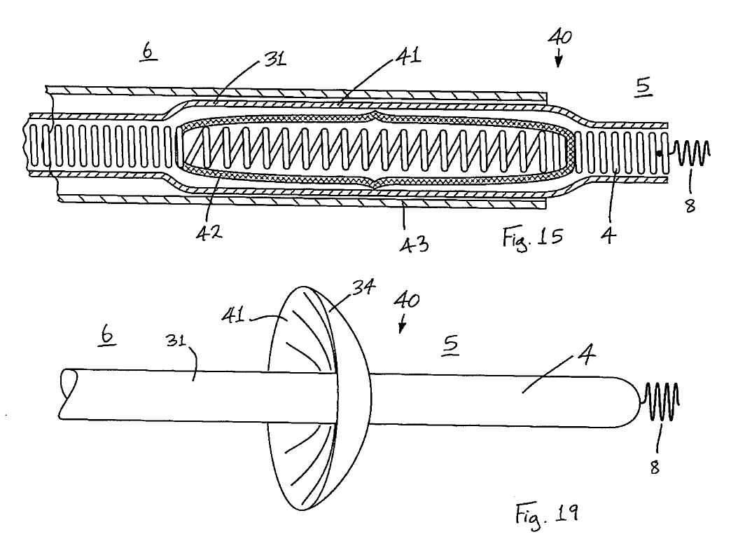

In this case the treatment element 41 comprises the membrane 31 as described previously with reference to FIGS. 11 to 13, and a biasing element 42. The biasing element 42 acts to bias the membrane 31 from the collapsed configuration (FIG. 14) towards the expanded configuration (FIGS. 17 and 18). In this case the biasing element 42 is of a shape-memory material, such as Nitinol.

A delivery catheter 43 is provided to retain the treatment element 41 in the collapsed configuration during delivery. The delivery catheter 43 houses the treatment element 41 during delivery (FIGS. 14 and 15).

In use, the collapsed treatment element 41 and the delivery catheter 43 are advanced over the support element 4 until the intermediate portion 34 of the treatment element 41 reaches the region of co-aptation of the valve leaflets 3 (FIG. 14). The delivery catheter 43 is then withdrawn proximally which enables the treatment element 41 to move from the collapsed configuration to the expanded configuration under the biasing action of the biasing element 42 (FIGS. 16 and 17).

FIGS. 14 to 19 illustrate the pacing lead 4 with the nitinol element 42. FIG. 15 illustrates the pacing lead 4 with more comprehensive construction detail and the nitinol support 42.

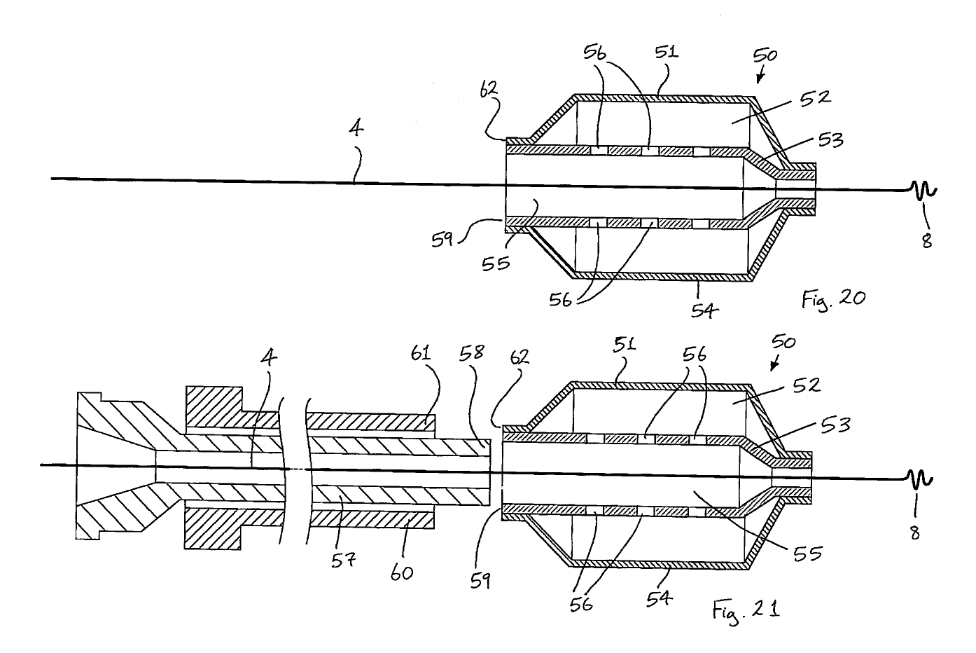

In FIGS. 20 to 29 there is illustrated another medical device 50 according to the invention, which is similar to the device 30 of FIGS. 11 to 13, and similar elements in FIGS. 20 to 29 are assigned the same reference numerals.

In this case the treatment element 51 is inflatable from the collapsed configuration (FIG. 26) to the expanded configuration (FIG. 27). The treatment element 51 comprises an annular-shaped inflatable region 52 defined between an inner tube 53 and an outer membrane 54, and a fluid passageway 55 to connect the inflatable region 52 in fluid communication with an inflation fluid source. A plurality of openings 56 are provided in the inner tube 53 to connect the inflatable region 52 in communication with the fluid passageway 55.

A delivery catheter 57 is provided to facilitate delivery of the treatment element 51 to the region of co-aptation of the valve leaflets 3. The distal end 58 of the delivery catheter 57 is coupled to the proximal end 59 of the inner tube 53 during delivery.

A release member 60 is also provided to facilitate decoupling of the treatment element 51 from the delivery catheter 57. The distal end 61 of the release member 60 is engagable with the proximal end 62 of the outer membrane 54 to decouple the treatment element 51 from the delivery catheter 57.

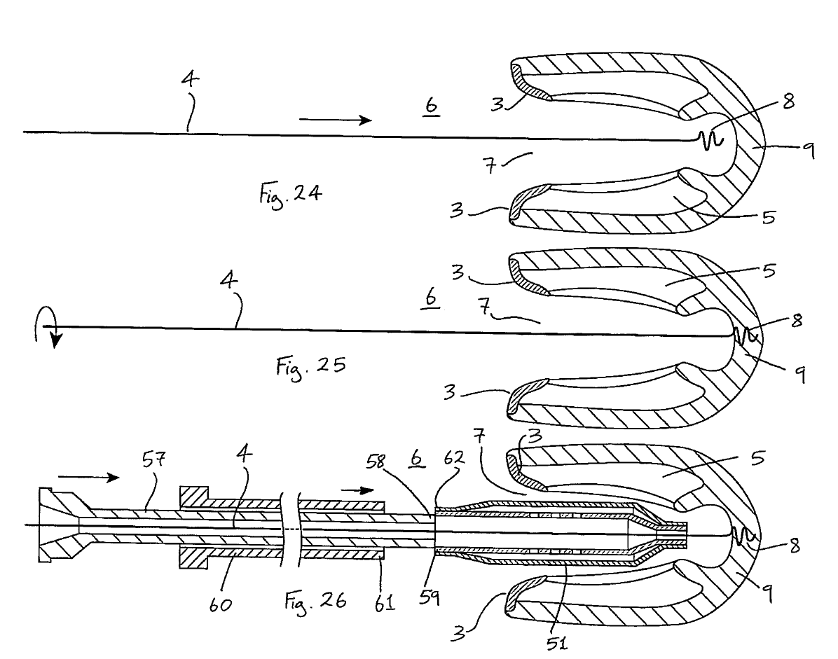

In use, the support element 4 is advanced through the atrium 6, through the valve opening 7, and into the ventricle 5 until the anchor element 8 reaches the apex 9 of the ventricle 5 (FIG. 24). The support element 4 is then rotated to screw the anchor element 8 into the ventricle wall at the apex 9 of the ventricle 5 (FIG. 25).

The delivery catheter 57 and the collapsed treatment element 51 are advanced together, with the treatment element 51 coupled to the delivery catheter 57, over the support element 4 until the treatment element 51 reaches the region of co-aptation of the valve leaflets 3 (FIG. 26). The inflatable region 52 is then inflated to move the treatment element 51 from the collapsed configuration to the expanded configuration (FIG. 27).

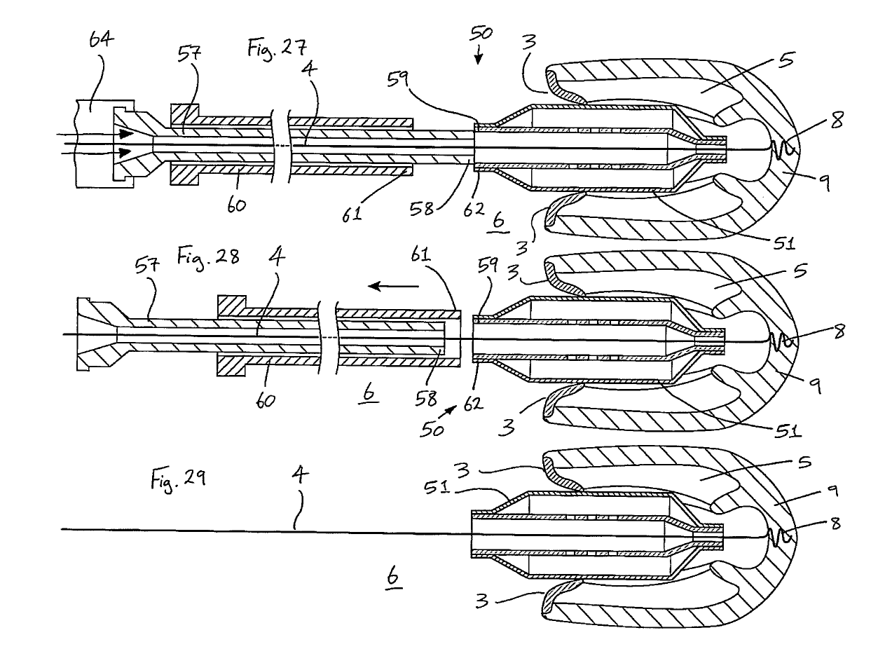

To decouple the treatment element 51 from the delivery catheter 57, the release member 60 is moved distally relative to the delivery catheter 57 to engage the distal end 61 of the release member 60 with the proximal end 62 of the outer membrane 54. The delivery catheter 57 and the release member 60 are then withdrawn from the atrium 6 (FIG. 28).

FIG. 21 shows a schematic representation of the catheter 57 suitable for use with the valve repair device 50. The catheter 57 has a proximal end and the distal end 58 and an inner tube that extends from the proximal end to the distal end 58. The inner tube has a wire lumen and an inflation lumen. In the embodiment shown in FIG. 21 a single lumen is used for the wire 4 and the inflation. It will be appreciated that a two lumen construction could also be used. The catheter 57 has a coupled configuration and a decoupled configuration (FIG. 21). In the coupled configuration the inner lumen(s) of the catheter 57 is in communication with the inner lumen 55 of the mounting tube 53. The catheter 57 also has the outer disengagement tube 60 for decoupling the repair element 50 from the catheter 57 after inflation. The disengagement tube 60 has a retracted position (FIG. 21) and an advanced position. In the retracted position, the repair device 50 can be coupled to the catheter 57, advanced over the wire mandrel 4 to its position of placement adjacent a defective valve, and expanded at that position. When the repair element 50 is positioned correctly the distal end 61 of the disengagement tube 60 is advanced relative to the inner tube 57 to decouple the repair device 50 from the catheter 57. The delivery catheter 57 also comprises an inflation adapter at its proximal end for engagement with inflation devices.

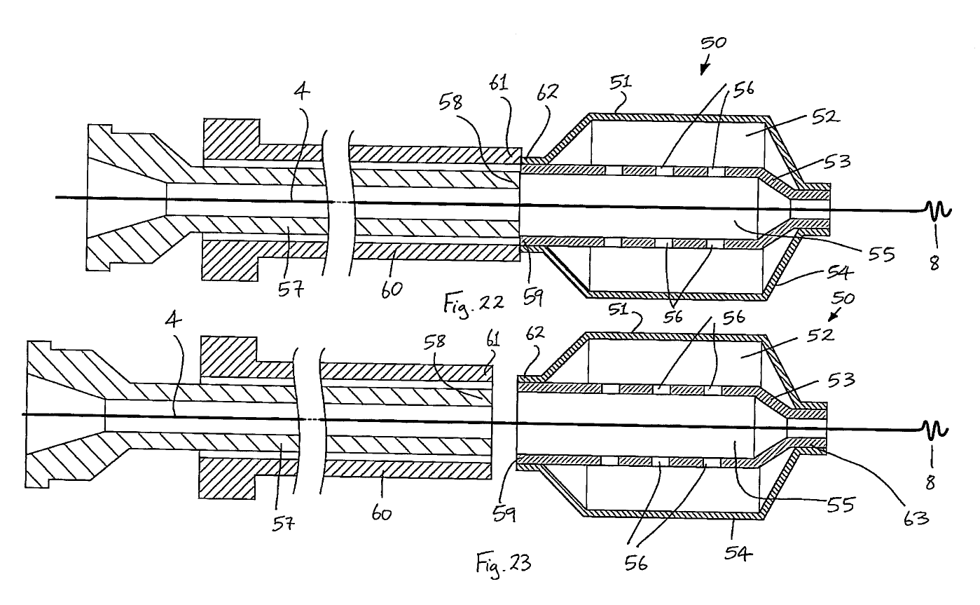

FIG. 22 shows the outer disengagement tube 60 in the retracted position with the repair element 50 and the catheter 57 coupled.

FIG. 23 shows the outer disengagement tube 60 in its advanced position with the repair element 50 decoupled from the catheter 57. The mounting tube 53 of the repair element 50 contains a neck down section 63 for frictional engagement with the wire mandrel 4.

### NUMBERING SYSTEM FIGS. 20-29

50 Repair element
53 Mounting tube
4 Wire mandrel
8 Anchor element
56 Inflation port
57 Delivery catheter
60 Disengagement tube
55 Mounting tube inner lumen
58 Delivery catheter distal end
5 Ventricle
9 Myocardium
51 Inflation device

FIG. 20 shows the device 50 for repairing a defective coronary valve. The device 50 comprises the inflatable membrane 54, and the mounting tube 53. The wire mandrel 4 has the wall anchor element 8 adjacent its distal end.

The valve arrangement comprises inflation ports 56. The device 50 has an expanded configuration and a collapsed configuration. In the collapsed configuration the device 50 can be delivered through a catheter and/or over a guidewire. In the expanded configuration the distal end of the inflatable membrane 54 is placed adjacent the defective coronary valve and restores the efficacy of the valve. The mounting tube 53 has the proximal end 59 and a distal end. The proximal end 59 is designed to couple with the delivery catheter 57. The diameter of the proximal end 59 of the mounting tube 53 is sufficient to allow fluid inflation of the membrane 54. The distal end of the mounting tube 53 is sized relative to the mounting mandrel 4. The gap between the distal end of the mounting tube 53 and the mounting mandrel 4 is sufficiently small to prevent significant fluid flow during inflation. The anchor element 8 adjacent the distal end of the mounting mandrel 4 is used to anchor the device 50 to the wall of the heart. The anchor element 8 comprises a cork screw feature that anchors to the myocardium 9 with a twisting action. It will be appreciated that other anchor arrangements are also possible.

The repair element 50 may be delivered with a number of different techniques, for example:

In a first method:

the wire mandrel 4 is advanced across the defective valve through a procedural catheter and anchored in the myocardium 9 of the ventricle 5;

the repair element 51 and the delivery catheter 57 are advanced over the wire 4 to the site of placement adjacent the valve;

the repair element 51 is expanded;

the repair element position is finely adjusted;

the delivery catheter 57 is removed.

In a second method:

the wire mandrel 4, the repair element 51 and the delivery catheter 57 are advanced through the procedural catheter together;

the repair element 51 is expanded adjacent the defective valve;

the efficacy of the repair element 51 is checked by evaluating regurgitation through the valve;

the wire mandrel 4 is anchored to the wall of the myocardium 9.

In a third method:

the wire mandrel 4, the repair element 51 and the delivery catheter 57 are advanced through the procedural catheter together;

the wire mandrel 4 is anchored to the wall of the myocardium 9;

the repair element 51 is expanded adjacent the defective valve;

the delivery catheter 57 is removed.

FIGS. 24 to 29 demonstrate one method of using the repair element 50 of FIGS. 20-23. In FIGS. 24 and 25 the wire mandrel 4 is advanced through a guide sheath (not shown) across the valve (mitral or tricuspid) and the tip 8 of the mandrel 4 is placed inside the ventricle 5. The tip of the wire mandrel 4 contains the anchor element 8 at its distal end. The mandrel 4 is further advanced and the anchor element 8 is embedded in the myocardium 9 of the ventricle 5. The anchoring step involves a cork screw action for the anchor 8.

The delivery of the repair element 51 is shown in FIG. 26. The repair element 51 is shown in its collapsed configuration with the membrane 54 wrapping to a low profile around the mounting tube 53. The repair element 51, and the delivery catheter 57 are advanced to the site of placement.

FIG. 27 shows the expansion of the membrane 54. In this embodiment the inflation means is pressurised fluid. It will be appreciated that the membrane 54 could be expanded with a variety of techniques including the use of stored elastic or shape memory energy. An inflation device 64 is connected to the inflation adaptor and pressurised fluid is delivered through the delivery catheter lumen to the lumen 55 of the mounting tube 53. The pressure of the fluid in the mounting tube 53 expands the repair element 51. The gap between the distal end of the mounting tube 53 and the wire mandrel 4 is small and little fluid can escape. Preferably the gap creates an interference fit such that fluid loss is negligible and a frictional fit is established between the repair element 51 and the wire mandrel 4.

FIG. 28 shows the decoupling of the repair element 51 from the catheter 57 through the advancing of the decoupling tube 60 relative to the inner tube 57. FIG. 29 shows the repair element 51 implanted with the delivery catheter 57 removed.

FIGS. 30 to 36 illustrate another medical device 70 according to the invention, which is similar to the device 50 of FIGS. 20 to 29, and similar elements in FIGS. 30 to 36 are assigned the same reference numerals.

In this case the support element 71 comprises a relatively small diameter, proximal, non-mounting region 72 and a relatively large diameter, distal, mounting region 73. During delivery to the region of co-aptation of the valve leaflets 3, the treatment element 51 is advanced over the non-mounting region 72 of the support element 71. The inner tube 53 of the treatment element 51 is engagable with the mounting region 73 of the support element 71 to mount the treatment element 51 to the mounting region 73 of the support element 71.

In this case a release member is not required to decouple the treatment element 51 from the delivery catheter 57.

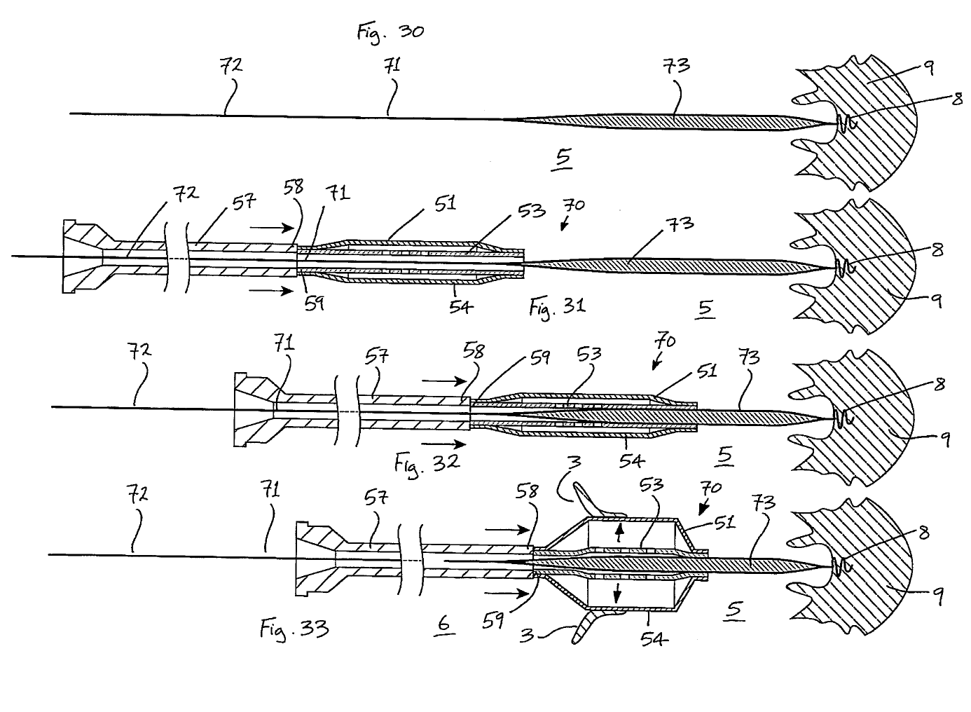

In use, the treatment element 51 is advanced over the non-mounting region 72 of the support element 71 (FIG. 31) until the inner tube 53 of the treatment element 51 engages with the mounting region 73 (FIG. 32). The engagement of the inner tube 53 with the mounting region 73 effectively couples the treatment element 51 to the support element 71. The expanded treatment element 51 may then be decoupled from the delivery catheter 57 by withdrawing the delivery catheter 57 (FIG. 35).

FIGS. 30-36 show the embodiment of the invention in which the repair element 70 is designed to have a small number of components and be as flexible as possible. The mounting tube 53 is soft and flexible and is preferably made from the same material as the inflatable membrane 54. The catheter 57 comprises an outer tube. This embodiment also features the profiled mandrel 71.

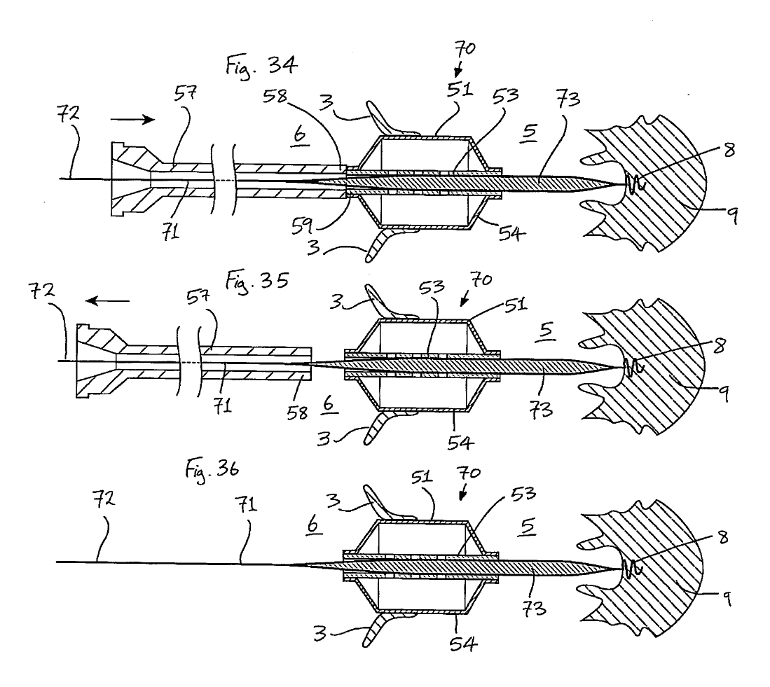

FIG. 30 shows the profiled mandrel 71 with the anchor element 8 at its distal end. The anchor element 8 is anchored in the myocardium 9 of the ventricle 5. The distal end 73 of the mandrel 71 has a profiled shape. In one embodiment this profiled shape comprises an enlarged segment 73 with a transition taper. The enlarged end 73 creates an interference fit between the mounting tube 53 and the profiled mandrel 73, as shown in FIG. 32. This frictional engagement prevents relative movement between the two after implantation (FIGS. 34 and 35) and this locks the repair element 51 relative to the anchor element 8.

FIGS. 37-40 show a series of possible designs suitable as profiled mandrels for use with this invention.

FIG. 37 shows a profiled mandrel 80 made from one homogenous material. The mandrel 80 is preferably a biocompatible material. Suitable polymers include the fluoropolymers, polyurethanes, polyesters especially PET, silicone based polymers. Preferred metallic materials include stainless steel and nitinol. Preferred metals may be electopolished.

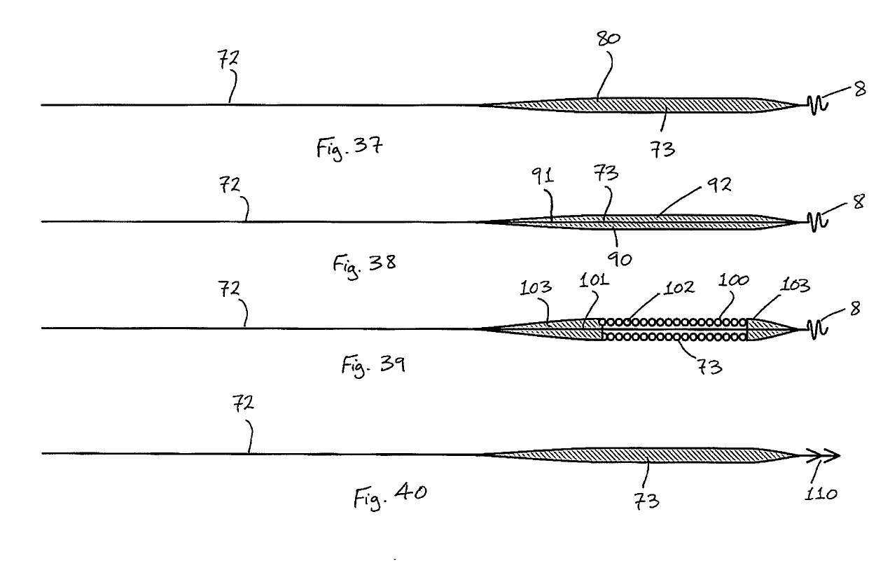

FIG. 38 shows a profiled mandrel 90 as a composite arrangement. In this embodiment the mandrel comprises a core 91 and an outer covering 92. The core 91 may be a metallic rod or tube while the outer covering 92 may be a polymer, or metallic tubular element. The polymers described above would be suitable. Metallic constructions may employ spring components.

FIG. 39 shows another profiled mandrel 100 composite arrangement. The mandrel 100 comprises a rod or tube 101 while the outer comprises a spring element 102 with transition components 103 at each end.

FIG. 40 shows an alternative anchoring system. The barbed arrangement 110 is easily inserted into the myocardium 9 but may be more difficult to remove. This anchor 110 could be used with any of the arrangements from FIGS. 37 to 39.

The invention is not limited to the embodiments hereinbefore described, with reference to the accompanying drawings, which may be varied in construction and detail.

1. A medical device suitable for use in treatment of a valve, the device comprising:—
a treatment element configured to be located at the region of co-aptation of leaflets of a valve to resist fluid flow in a retrograde direction through an opening of the valve;
at least one support element to support the treatment element at the region of co-aptation of the valve leaflets; and
at least one anchor element to anchor the at least one support element to a wall of body tissue;
the at least one anchor element being located at the distal end of the at least one support element;
the proximal end of the at least one support element being unconstrained relative to the body tissue wall.

2. A device as claimed in claim 1 wherein the support element is configured to extend through a valve opening.

3. A device as claimed in claim 1 wherein the anchor element is extendable into a body tissue wall.

4. A device as claimed in claim 1 wherein the anchor element is configured to releasably anchor the support element to a wall of body tissue.

5. A device as claimed in claim 1 wherein the anchor element comprises a threaded element.

6. A device as claimed in claim 1 wherein the anchor element is configured to anchor the support element to a ventricle of a heart.

7. A device as claimed in claim 1 wherein the proximal end of the support element is configured to be located externally of a heart.

8. A device as claimed in claim 1 wherein the treatment element is movable between a collapsed configuration and an expanded configuration.

9. A device as claimed in claim 8 wherein the treatment element is substantially tubular-shaped in the collapsed configuration.

10. A device as claimed in claim 8 wherein the treatment element is substantially disc-shaped in the expanded configuration.

11. A device as claimed in claim 8 wherein the treatment element is substantially curved in longitudinal cross-section in the expanded configuration.

12. A device as claimed in claim 1 wherein the treatment element is substantially non-circular in lateral cross-section.

13. A device as claimed in claim 8 wherein the treatment element is biased towards the expanded configuration.

14. A device as claimed in claim 8 wherein a first end of the treatment element is movable relative to a second end of the treatment element to move the treatment element between the collapsed configuration and the expanded configuration.

15. A device as claimed in claim 1 wherein the treatment element comprises a membrane.

16. A device as claimed in claim 8 wherein at least part of the treatment element is inflatable to move the treatment element from the collapsed configuration to the expanded configuration.

17. A device as claimed in claim 1 wherein the treatment element is movable between a delivery configuration and a deployed configuration.

18. A device as claimed in claim 1 wherein the treatment element comprises a collar member with a lumen extending therethrough.

19. A device as claimed in claim 1 wherein the treatment element is fixed to the support element.

20. A device as claimed in claim 1 wherein the treatment element is movable relative to the support element for delivery of the treatment element over the support element to the region of co-aptation of leaflets of a valve.

21. A device as claimed in claim 1 wherein the treatment element is mountable on the support element.

22. A device as claimed in claim 1 wherein the support element is substantially flexible.

23. A device as claimed in claim 1 wherein the support element comprises a wire element.

24. A device as claimed in claim 1 wherein the device comprises a delivery member coupleable to the treatment element to facilitate delivery of the treatment element to the region of co-aptation of leaflets of a valve.

25. A method of treating a valve, the method comprising the steps of:
locating a treatment element at the region of co-aptation of leaflets of the valve to resist fluid flow in a retrograde direction through an opening of the valve,
using at least one support element to support the treatment element at the region of co-aptation of the valve leaflets,
anchoring the distal end of the at least one support element to a wall of body tissue with the proximal end of the at least one support element being unconstrained relative to the body tissue wall.

26. A method as claimed in claim 25 wherein the support element extends through the valve opening.

27. A method as claimed in claim 25 wherein the distal end of the support element is extended into the body tissue wall.

28. A method as claimed in claim 27 wherein the distal end of the support element is extended only partially through the body tissue wall.

29. A method as claimed in claim 27 wherein the distal end of the support element is extended into the body tissue wall from an interior side of the body tissue wall.

30. A method as claimed in claim 25 wherein the distal end of the support element is releasably anchored to the body tissue wall.

31. A method as claimed in claim 25 wherein the distal end of the support element is anchored to a ventricle of a heart.

32. A method as claimed in claim 25 wherein the proximal end of the support element is located externally of a heart.

33. A method as claimed in claim 25 wherein the method comprises the step of moving the treatment element between a collapsed configuration and an expanded configuration.

34. A method as claimed in claim 33 wherein a first end of the treatment element is moved relative to a second end of the treatment element to move the treatment element between the collapsed configuration and the expanded configuration.

35. A method as claimed in claim 33 wherein at least part of the treatment element is inflated to move the treatment element from the collapsed configuration to the expanded configuration.

36. A method as claimed in claim 25 wherein the method comprises the step of moving the treatment element between a delivery configuration and a deployed configuration.

37. A method as claimed in claim 25 wherein the treatment element is fixed to the support element, and the method comprises the step of advancing the support element to deliver the treatment element to the region of co-aptation of the valve leaflets.

38. A method as claimed in claim 25 wherein the method comprises the step of moving the treatment element over the support element to deliver the treatment element to the region of co-aptation of the valve leaflets.

39. A method as claimed in claim 25 wherein the method comprises the step of mounting the treatment element to the support element.

40. A method as claimed in claim 25 wherein the method comprises the step of coupling the treatment element to a delivery member before delivery of the treatment element to the region of co-aptation of the valve leaflets.
